(19) United States
(12) Patent Application Publication
(10) Pub. No.: US 2024/0255425 A1
(43) Pub. Date: Aug. 1, 2024

(54) MARKER, METHOD AND DEVICE FOR ANALYZING A BIOLOGICAL SAMPLE

(71) Applicant: LEICA MICROSYSTEMS CMS GMBH, Wetzlar (DE)

(72) Inventors: Soeren ALSHEIMER, Wetzlar (DE); Kai WALTER, Wetzlar (DE); Joachim BRADL, Wetzlar (DE)

(21) Appl. No.: 18/290,315

(22) PCT Filed: Dec. 23, 2021
(86) PCT No.: PCT/EP2021/087558

§ 371 Date: Nov. 13, 2023

(30) Foreign Application Priority Data
May 19, 2021 (WO) PCT/EP2021/063310
Jun. 18, 2021 (WO) PCT/EP2021/066645
Aug. 28, 2021 (WO) PCT/EP2021/073819

(51) Int. Cl. G01N 21/64; C12Q 1/44

(52) U.S. Cl. CPC G01N 21/6428; C12Q 1/44; G01N 21/6458; G01N 2021/6419; G01N 2021/6441; G01N 2333/922

(57) ABSTRACT

A marker for marking a predetermined structure within a biological sample includes a marker base having an affinity reagent, and an attachment structure connected to the affinity reagent having two attachment sites. The attachment structure includes a cleavage site arranged between the attachment sites. The attachment structure is capable of being cut at the cleavage site by a cleaving agent in order to remove an attachment site from the marker base. The marker further includes at least two reporters. Each reporter includes a linker structure having a complementary attachment site configured to attach to one of the attachment sites, and a combination of at least two different fluorescent dyes. The combination of the at least two different fluorescent dyes is unique for each reporter. The complementary attachment site is unique for each reporter and configured such that each reporter attaches to a different attachment site of the marker base.

### CROSS REFERENCE TO RELATED APPLICATIONS

This application is a U.S. National Phase application under 35 U.S.C. § 371 of International Application No. PCT/EP2021/087558, filed on Dec. 23, 2021, and claims benefit to International Patent Application No. PCT/EP2021/073819, filed on Aug. 28, 2021 and International Patent Application No. PCT/EP2021/066645, filed on Jun. 18, 2021 and International Patent Application No. PCT/EP2021/063310, filed on May 19, 2021. The International Application No. PCT/EP2021/087558 was published in English on Nov. 24, 2022 as WO 2022/242896 A1 under PCT Article 21(2).

### FIELD

Embodiments of the present invention relate to marker for marking a predetermined structure within a biological sample. Embodiments of the present invention also relate to a method for analyzing a biological sample, and a device for analyzing a biological sample.

### BACKGROUND

In order to address key problems in the field of life sciences it is vital to precisely identify and to locate certain structures within biological samples, e.g. tissue samples or cell cultures. This can be done by introducing markers into the sample that bind to specific structures, e.g. specific biomolecules. These markers typically comprise an affinity reagent that attaches to the structure in question and one or more fluorescent dyes that are either directly conjugated to the affinity reagent or attached to the affinity reagent by other means, for example a secondary affinity reagent.

Fluorescence microscopy for example allows for imaging the sample with high spatial resolution but involves only a low number of different fluorescent dyes, typically between 1 and 5. The available dyes have to be distributed to all markers that are used to identify cell types, functional markers like protein-of-interest and general morphological markers in the same experiment. This means that cell types in most imaging experiments are merely poorly identified. While modern approaches that allow for a much more reliable and robust identification of cell types, e.g. based on the analysis of genetic regulatory networks (GRNs), exist they require a much higher number of different markers to be read-out from the sample.

The documents PCT/EP2021/063310 and PCT/EP2021/073819 propose markers and methods each for increasing the number of markers that can be used in a single fluorescence microscopy experiment. Each marker comprises a unique combination of dyes forming a code, that in principle identifies the respective marker. However, certain ambiguities remain, in particular when a large number of markers is in close proximity of each other. In order to resolve these ambiguities, it is necessary to remove the markers and repeat the image acquisition with a different set of markers. This process is time consuming and expensive.

### SUMMARY

Embodiments of the present invention provide a marker for marking a predetermined structure within a biological sample. The marker includes a marker base having an affinity reagent configured to attach to the predetermined structure of the sample, and an attachment structure connected to the affinity reagent having at least two attachment sites. The attachment structure includes at least one cleavage site arranged between the two attachment sites. The attachment structure is capable of being cut at the at least one cleavage site by a cleaving agent in order to remove at least one attachment site from the marker base. The marker further includes at least two reporters. Each reporter includes a linker structure having a complementary attachment site configured to attach to one of the two attachment sites of the attachment structure, and a combination of at least two different fluorescent dyes arranged on the linker structure. The combination of the at least two different fluorescent dyes is unique for each reporter. The complementary attachment site is unique for each reporter and configured such that each reporter attaches to a different attachment site of the marker base.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view of a marker for marking a predetermined structure within a biological sample according to an embodiment;

FIG. 2 is a schematic view of the marker according to FIG. 1 after the introduction of a first cleaving agent according to an embodiment;

FIG. 3 is a schematic view of the marker according to FIGS. 1 and 2 after the introduction of a second reporter according to an embodiment;

FIG. 4 is a schematic view of the marker according to FIGS. 1 to 3 after the introduction of a second cleaving agent according to an embodiment;

FIG. 5 is a schematic view of the marker according to FIGS. 1 to 4 after the introduction of a third reporter according to an embodiment;

FIG. 6 is a flowchart of the method for analyzing the biological sample utilizing the marker described above with reference to FIGS. 1 to 5 according to an embodiment; and

FIG. 7 shows a schematic drawing of a device for analyzing a biological sample according to an embodiment.

### DETAILED DESCRIPTION

Embodiments of the present invention provide a marker for marking a predetermined structure within a biological sample, a method for analyzing a biological sample and a device for analyzing a biological sample that allows for the reliable identification of a high number of predetermined structure at a low cost and time expenditure.

According to some embodiments, the marker for marking a predetermined structure within a biological sample comprises a marker base. The marker base has an affinity reagent configured to attach to the predetermined structure of the sample, and an attachment structure connected to the affinity reagent having at least two attachment sites. The attachment structure comprises at least one cleavage site arranged between the attachment sites. The attachment structure can be cut at the at least one cleavage site by a cleaving agent in order to remove at least one attachment site from the marker base. The marker further comprises at least two reporters, each reporter comprising a linker structure having a complementary attachment site configured to attach to one of the attachment sites, and a combination of at least two different fluorescent dyes arranged on the linker structure. The combination of dyes is unique for each reporter. The complementary attachment site is unique for each reporter and configured such that each reporter attaches to a different attachment site of the marker base.

The affinity reagent may in particular be an antibody, a single-domain antibody (also known as nanobody), a combination of at least two single-domain antibodies, an aptamer, an oligonucleotide, a morpholino, a PNA complementary to a predetermined RNA, DNA target sequence, a ligand (for example a drug or a drug-like molecule), or a toxin, for example a Phalloidin a toxin that binds to an actin filament. The predetermined structure may be a specific bio-molecule, for example a protein, an RNA sequence, a peptide, a DNA sequence, a metabolite, a hormone, a neurotransmitter, a vitamin or a micronutrient. The predetermined structure may also by a single analyte, for example a metal ion, in particular a heavy metal ion such as Cd(II), Co(II), Pb(II), Hg(II) or U(VI).

The affinity reagent of the marker base attaches itself to the predetermined structure when it is introduced into the sample. The predetermined structure is also called an analyte or target or target molecule. Since the affinity reagent is connected to the attachment structure, the attachment structure is connected to the predetermined structure as well. Thereby, the marker base allows the different reporters to be attached to the predetermined structure. Each reporter comprises a unique combination of dyes forming a code, that identifies the respective reporter. Thereby, the predetermined structure is made visible to fluorescence imaging.

The cleavage site is arranged along the linker structure between the attachment sites. By cutting the attachment structure at the cleavage site, one of the attachment sites is removed from the attachment structure. When one of the reporters is connected to the removed attachment site, the reporter is removed as well. This means that the removed reporter is not connected to the predetermined structure anymore and can be washed out of the sample. After the removed reporters have been washed out, another reporter that attaches itself to the remaining attachment site may be introduced to the sample. Thus, the marker allows the predetermined structure to be marked or encoded sequentially in a cyclic or round-based fashion. Thereby, ambiguities arising in a first round of marking can be resolved in later rounds of marking without necessitating the removal or reintroduction of affinity reagents. This in turn reduces cost and time expenditure for experiments involving a high number markers, i.e. a high number of predetermined structures to be identified.

In a preferred embodiment, the linker structures are formed by oligonucleotides or peptides. The linker structure holds the fluorescent dyes and allows them to connect to the attachment site. The fluorescent dyes may be directly, i.e. covalently, or indirectly, for example by an affinity tag-affinity ligand combination, coupled to the linker structure. The linker structure itself may comprise one or more oligonucleotides, for example DNA, RNA, LNA, PNA, a morpholino or another artificial oligonucleotide. The linker structure may also comprise peptides or other DNA- or RNA-analogues. Oligonucleotides and peptides alike can be synthesized to suit specific needs by standardized methods. The reporters can thus be produced easily and cost-effectively.

In another preferred embodiment, the cleavage site is an enzymatic cleavage site and the attachment structure can be cut at the cleavage site by an enzymatic cleaving agent. Preferably, the enzymatic cleavage site is a target site of a restriction enzyme, a CRISPR/Cas target, a recombinase target site, in particular a loxP site or a flippase target site, or a caspase target site. In order to remove one or more of the reporters from the marker, the enzymatic cleaving agent is introduced into the sample. After the enzymatic cleaving agent has cut the attachment structure at the cleavage site, the cut of part of the attachment structure and the reporter connected to this part of the attachment structure can be washed out of the sample. Enzymatic cleaving has the advantage of targeting only the cleavage site, thereby reducing damage to other structures.

In another preferred embodiment, the cleavage site is a photocleavage site and the attachment structure can be cut at the cleavage site by photolysis, in particular by UV light. Cutting the attachment structure by means of photolysis is very efficient and easy to automate.

In another preferred embodiment, the attachment sites and/or the complementary attachment sites are oligonucleotides. Oligonucleotides can easily be made into a unique bar code like structure. Thus, each attachment site and its respective complementary attachment site can be made such, that a particular reporter only attaches itself to a specific attachment site.

The invention also relates to a method for analyzing a biological sample, comprising the following steps: a) Providing at least one marker as described above. b) Introducing the marker base and the first reporter into the sample into the sample. c) Directing at least one first excitation light onto the sample in order to excite the fluorescent dyes of the first reporter. d) Generating at least one first readout from fluorescence light emitted by excited fluorescent dyes located in a readout volume of the sample. e) Introducing at least one cleaving agent into the sample in order to remove the first reporter from the marker base. f) Introducing the second reporter into the sample. g) Directing at least one second excitation light onto the sample in order to excite the fluorescent dyes of the second reporter. h) Generating at least one second readout from fluorescence light emitted by excited fluorescent dyes located in the readout volume of the sample. i) Determining whether the marker is present in the readout volume based on the first and second readouts.

The first reporter may already be attached to the attachment structure of the marker base when the marker is introduced into the sample. Alternatively, the marker base and the first reporter are introduced separately into sample. The first excitation light may comprise light of a single wavelength or wavelength spectrum depending on the specific fluorescent dyes of the first reporter. More than one first excitation light may be used, for example light emitted by different light sources either simultaneously or sequentially. The cleaving agent may be an enzymatic cleaving agent in case the cleavage sites are targets for said enzymatic cleaving agent. Alternatively, the cleaving agent may be light, in particular UV-light, in case the cleavage sites can be cut by photolysis.

It is noted that in particular when the method for analyzing the biological sample is applied, the marker according to claim 1 preferably comprises at least one reporter comprising a linker structure having a complementary attachment site being unique for the at least one reporter and being configured to attach to one of the attachment sites, and a combination of at least two different fluorescent dyes arranged on the linker structure, wherein the combination of dyes is unique for the at least one reporter. Subsequently, preferably after the steps a) to e) of the method mentioned above have been applied, the marker comprises at least one further (e.g. second) reporter having a complementary attachment site being unique for the at least one further (e.g. second) reporter and being configured to attach to a different attachment site, and a combination of at least two different fluorescent dyes arranged on the linker structure, wherein the at least one further reporter is adapted to be attached to the different attachment site. It therefore might be suitable to configure the marker in at least two parts, the first part comprising the marker base having the affinity reagent and the attachment structure connected to the affinity reagent having at least two attachment sites, wherein the attachment structure comprises at least one cleavage site arranged between the attachment sites and the at least one reporter. The second (and any further) part of the marker might comprise the at least one further (e.g. second) reporter having a complementary attachment site being unique for the at least one further (e.g. second) reporter and being configured to attach to a different attachment site, and a combination of at least two different fluorescent dyes arranged on the linker structure, wherein the at least one further reporter is adapted to be attached to the different attachment site.

The method uses one or more markers as described above in order to analyze the sample. The markers are used to target predetermined structures in the sample in order to make them visible and identifiable. Individual markers are identified by their attached reporters by means of the first and second readouts which comprise information about the emitted fluorescence light, in particular an emission spectrum, a fluorescence emission intensity or a fluorescence lifetime or an excitation fingerprint of the fluorescent dyes. Since at least two different readouts are generated and for the generation of each readout, different reporters are used, ambiguities arising during the capture of the first readout can be resolved in subsequent readouts. This allows for the reliable identification and separation of a high number of markers, and thus a high number of predetermined structures to be identified.

In a preferred embodiment, the method comprises the further step of cross-linking the affinity reagent of the marker to the predetermined structure with a cross-linking agent. The cross-linking agent, for example glutaraldehyde, stabilizes the interaction between the affinity reagent and the predetermined structure. Thereby, more cycles of introducing and removing reporters can be performed without the bond between the affinity reagent and the predetermined structure breaking.

In another preferred embodiment, generating the first and/or second readouts comprises separating the emission light emitted by the excited fluorescent dyes into detection channels. The detection channels correspond to at least one emission characteristic of the fluorescent dyes. The emission characteristic is one of the following: an emission spectrum, a fluorescence intensity, and a fluorescence lifetime. Preferably, the marker is provided such that each fluorescent dye of the first and second reporter corresponds to one detection channel of the first and second readout, respectively. The channels are generated in order to separate the contributions of the different fluorescent dyes to a single readout. Generating the channels may comprise at least one of spectral unmixing, a determination of a fluorescence lifetime and determination of an excitation fingerprint of the fluorescent dyes. Spectral unmixing (also referred to as spectral imaging and linear unmixing, or channel unmixing) may be performed in various ways including but not limited to linear unmixing, principle component analysis, learning unsupervised means of spectra, support vector machines, neural networks, (spectral) phasor approach, and Monte Carlo unmixing algorithm. In order to reduce crosstalk between the fluorescent dyes associated with different markers, several of these techniques may be employed. The unmixing techniques can also be used to separate contributions from different fluorescent dyes, i.e. the crosstalk due to overlapping emission spectra, in for example a single pixel of a readout. Employing these techniques can greatly enhance the sensitivity of the method due to reduced noise. Further, the fluorescence lifetime and the excitation fingerprint of a fluorescent dye can be used in order to correctly identify fluorescent dyes, and thus reporters. This can be used to employ more sets of markers per readout. In turn, this vastly increases the overall number of markers that can be used in a single experiment.

In another preferred embodiment, all fluorescent dyes of the reporters are divided into sets of dyes. Each fluorescent dye in the same set can be excited by essentially one wavelength spectrum or by the same wavelength spectrum. In steps c) and g) at least one excitation light for each set of dyes is directed at the sample in order to excite the fluorescent dyes of the respective set. In steps d) and h) at least one readout for each set of dyes is generated from fluorescence light emitted by the excited fluorescent dyes located in the readout volume of the sample. Each set of fluorescent dyes may be excited independently by a different excitation light and, thus, fluorescence light emitted by each set may also be detected independently. This is used to generate multiple readouts, each capturing fluorescence light emitted by a different set. In each readout different fluorescent dyes are excited allowing a more robust identification of the different dyes, and thus the reporters and associated predetermined structures of the sample.

In another preferred embodiment, in steps c) and d) the excitation lights are directed onto the sample in a sequence temporally following each other. Preferably, the time between applying the different excitation lights is longer than the fluorescence lifetime of the fluorescent dyes. Thereby, crosstalk between different fluorescent dyes can be reduced and the sensitivity of the method is further improved.

In another preferred embodiment, the first reporter is washed out of the sample before the second excitation light is directed onto the sample. Washing out unbound reporters ensures that only markers that are actually attached to their associated predetermined structure are detected. Thus, washing out unbound reporters prevents misidentification of structures in the sample and makes the method more reliable.

In another preferred embodiment, the first and/or second readout comprises at least one image of the readout volume or a readout image data stream of the readout volume. In particular, the method comprises the further step of capturing a hyperspectral image of the sample in order to generate the first and/or second readouts. In contrast to multispectral imaging, which captures a limited number of wavelength bands, typically less than or around 10, a hyperspectral image captures tens or hundreds of wavelength bands per pixel. In other words, hyperspectral images have a very high spectral resolution. This allows for a much finer differentiation of fluorescent dyes based on their emission spectrum and thereby increases the sensitivity and reliability of the method.

In another preferred embodiment, the method comprises the further step of stabilizing the fluorescence lifetime of at least one fluorescent dye, in particular by placing the fluorescent dye in a shielded environment by at least one of encapsulating, polymer-matrix embedding, and co-crystallizing. Stabilizing the fluorescence lifetime allows for the much more reliable identification of the stabilized fluorescent dyes based on their lifetime. The method can also be used to increase the fluorescence lifetime of some fluorescent dyes of otherwise equal fluorescent dyes, providing a further differentiating feature, and thereby increasing the number of fluorescent dyes that can be used in a single experiment.

The invention further relates to a device for analyzing a biological sample. The device is adapted to carry out the method described above.

The device has the same advantages as the method described above and can be supplemented using the features of the dependent claims directed at the method.

In a preferred embodiment, the device comprises a microscope, a plate reader, a cytometer, an imaging cytometer, or a fluorescence activated cell sorter configured to generate the first and second readouts. The microscope is preferably a lens-free microscope, a light field microscope, a widefield microscope, a fluorescence widefield microscope, a light sheet microscope, a scanning microscope, a spinning disc microscope or a confocal scanning microscope.

In another preferred embodiment, the device is configured to determine at least one of the following: a fluorescence emission intensity, a fluorescence lifetime, a value representing a fluorescence lifetime, an emission spectrum, an excitation fingerprint, and a fluorescence anisotropy of the fluorescent dyes.

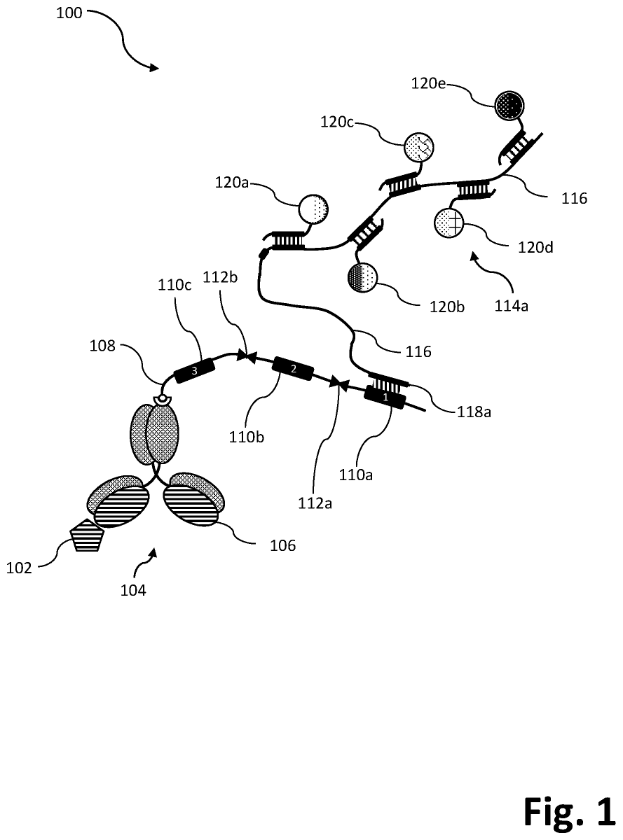

FIG. 1 is a schematic view of a marker 100 for marking a predetermined structure 102 within a biological sample according to an embodiment.

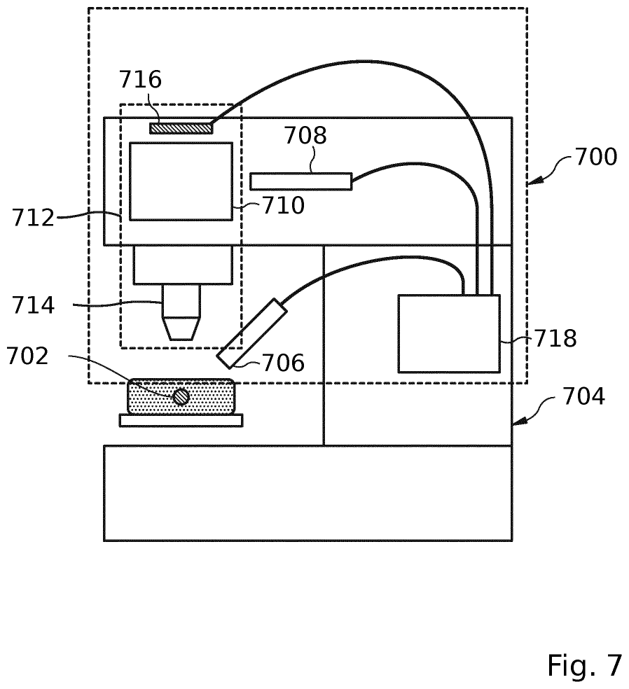

The predetermined structure 102 may be a specific bio-molecule or a single analyte, for example a metal ion, located within a sample 702 (c.f. FIG. 7). In FIG. 1 the predetermined structure 102 is shown as a pentagon.

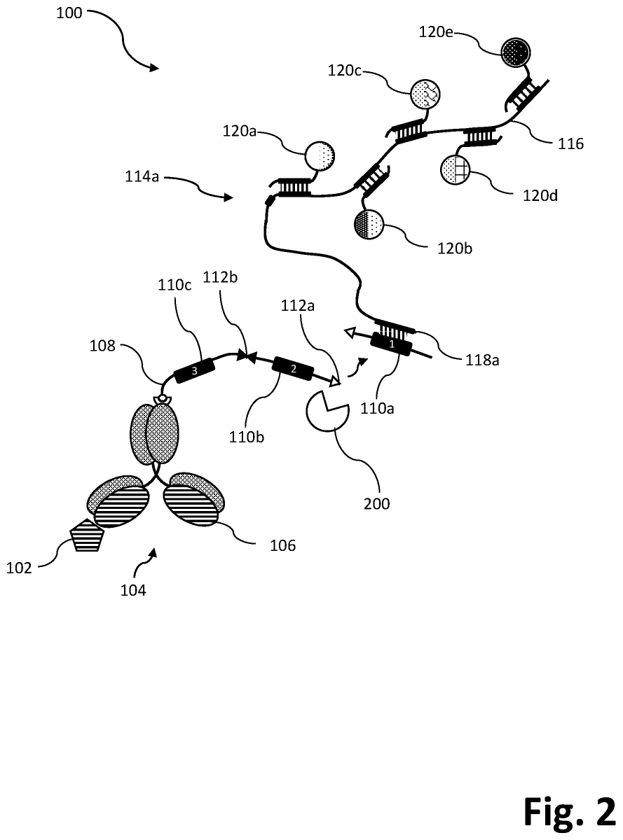

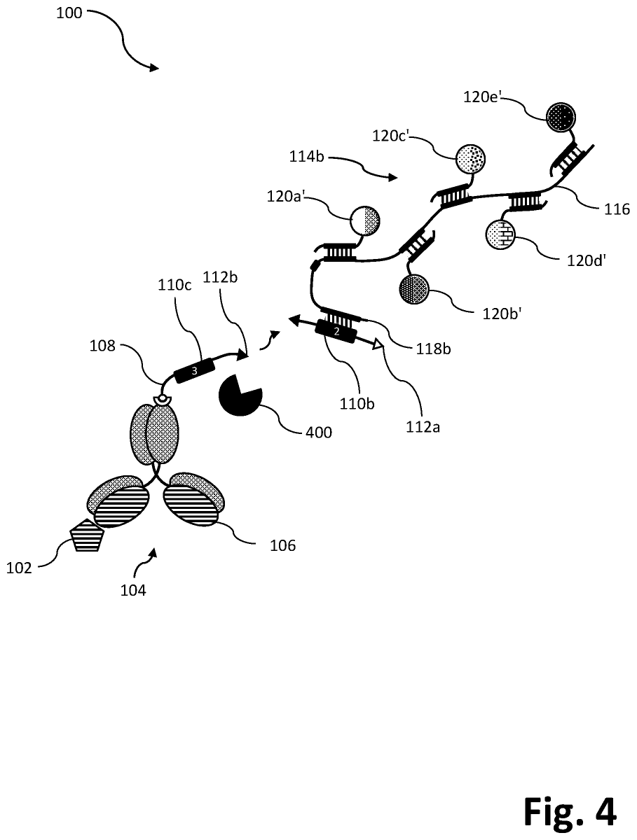

The marker 100 has a marker base 104 comprising an affinity reagent 106 and an attachment structure 108 connected to the affinity reagent 106. The affinity reagent 106 is configured to attach itself to the predetermined structure 102, thereby connecting the attachment structure 108 to the predetermined structure 102. A cross-linking agent, for example glutaraldehyde, may be used to strengthen the bond between the affinity reagent 106 and the predetermined structure 102. The attachment structure 108 is exemplary formed as long chain along which three attachment sites 110a, 110b, 110c are formed. Each attachment site 110a, 110b, 110c comprises a different molecule that is unique to the attachment site 110a, 110b, 110c. For example, the attachment site 110a, 110b, 110c may be formed by an oligomer such as an oligonucleotide comprising a unique sequence of nucleotides. Two cleavage sites 112a, 112b are arranged between the three attachment sites 110a, 110b, 110c. The attachment structure 108 can be cut at the two cleavage sites 112a, 112b by cleaving agents 200, 400 (c.f. FIGS. 2 and 4) in order to remove attachment sites 110a, 110b, 110c from the attachment structure 108. When a first cleavage site 112a is cut by a first cleaving agent 200, a first attachment site 110a is removed. When a second cleavage site 112b is cut by a second cleaving agent 400, a second attachment site 110b is removed and only a third attachment site 110c remains connected to the marker base 104. The cutting of the attachment structure 108 is described in more detail below with reference to FIGS. 2 and 4.

The marker 100 further comprises reporters 114a, 114b, 114c one of which is shown in FIG. 1. The reporters 114a, 114b, 114c comprise a linker structure 116 exemplary formed as a long chain. The linker structure 116 comprises a complementary attachment site 118a configured to attach to a first attachment site 110a of the attachment structure 108. The complementary attachment site 118a comprises a molecule that is complementary the molecule of the associated attachment site 110a. Along the linker structure 116, fluorescent dyes 120a, 120b, 120c, 120d, 120e are arranged. In the present embodiment, the fluorescent dyes 120a, 120b, 120c, 120d, 120e are indirectly bound to the linker structure 116 by means of a secondary structure. In FIG. 1, the fluorescent dyes 120a, 120b, 120c, 120d, 120e are bound to oligonucleotide sequences that are attached to complementary oligonucleotide sequences arranged on the linker structure 116. However, the fluorescent dyes 120a, 120b, 120c, 120d, 120e may also be directly, i.e. covalently bound to the linker structure 116. The combination of fluorescent dyes 120a, 120b, 120c, 120d, 120e of each of the reporters 114a, 114b, 114c is unique to the respective reporter 114a, 114b, 114c. This allows the reporters 114a, 114b, 114c to be uniquely identified. By using a combination of fluorescent dyes 120a, 120b, 120c, 120d, 120e instead of a single dye, the number of reporters 114a, 114b, 114c that can be uniquely identified is vastly increased. For example, 10 different fluorescent dyes are used. There are 45 unique two-dye combinations and 252 unique five-dye combinations. This means, that up to 252 different predetermined structures 102 can be marked and uniquely identified with 10 fluorescent dyes.

FIG. 2 is a schematic view of the marker 100 according to FIG. 1 after the introduction of the first cleaving agent 200.

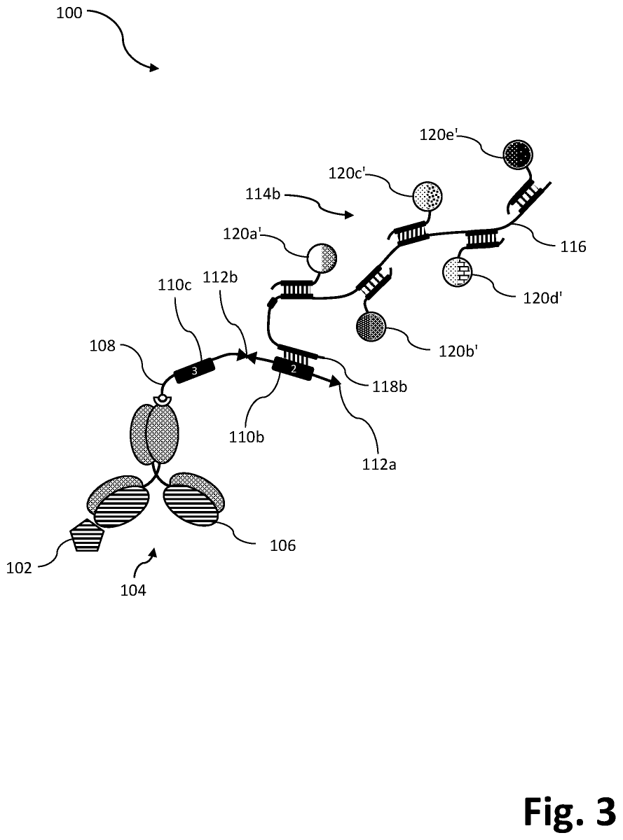

In the present embodiment, the cleaving agents 200, 400 are enzymatic cleaving agents. Alternatively, the cleaving agents may also be light, in particular UV-light, that cuts the attachment structure 108 at the cleavage sites 112a, 112b by means of photolysis. In FIG. 2, the attachment structure 108 is cut between the first and second attachment sites 110a, 110b by the first cleaving agent 200. Thereby, the first attachment site 110a and the first reporter 114a are removed from the marker 100. The removed reporter 114a may then be washed out of the sample 702. In a next step, a second reporter 114b (c.f. FIG. 3) may be introduced into the sample 702. This is described below with reference to FIG. 3.

FIG. 3 is a schematic view of the marker 100 according to FIGS. 1 and 2 after the introduction of the second reporter 114b.

The second reporter 114b comprises a different combination of fluorescent dyes 120a′, 120b′, 120c′, 120d′, 120e′ than the first reporter 114a. The complementary attachment site 118b of the second reporter 114b is configured to attach itself to the second attachment site 110b of the attachment structure 108. Accordingly, the second reporter 114b is attached to the second attachment site 110b in FIG. 3.

FIG. 4 is a schematic view of the marker 100 according to FIGS. 1 to 3 after the introduction of the second cleaving agent 400.

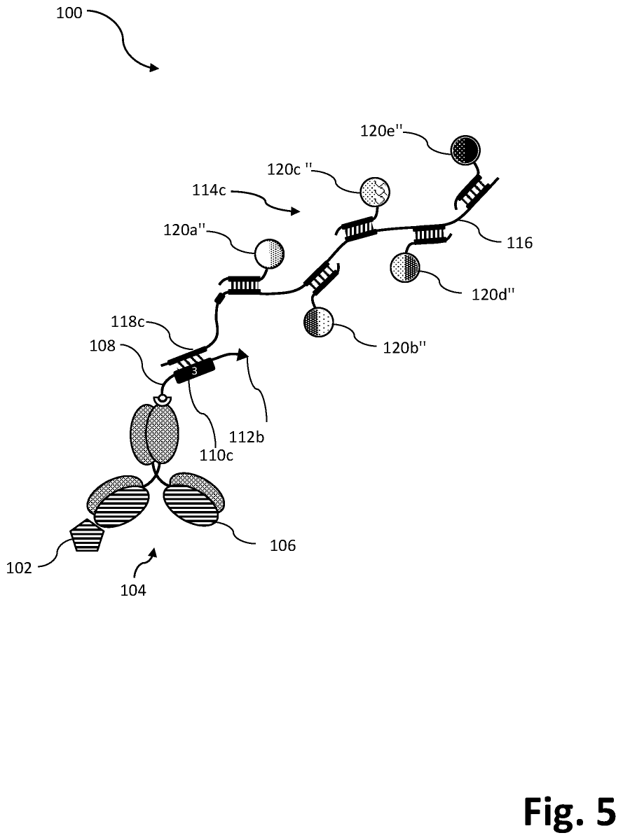

In FIG. 4, the attachment structure 108 is cut again by the second cleaving agent 400. The cut is made between the second and third attachment sites 110b, 110c, thereby removing the second attachment site 110b and the second reporter 114b from the marker base 104. The removed second reporter 114b may then be washed out of the sample 702. In a next step, a third reporter 114c (c.f. FIG. 5) may be introduced into the sample 702. This is described below with reference to FIG. 5.

FIG. 5 is a schematic view of the marker 100 according to FIGS. 1 to 4 after the introduction of the third reporter 114c.

The third reporter 114a comprises yet another different combination of fluorescent dyes 120a″, 120b″, 120c″, 120d″, 120e″ than the first and second reporters 114a, 114b. The complementary attachment site 118c of the third reporter 114c is configured to attach itself to the third attachment site 110c of the attachment structure 108. Accordingly, the third reporter 114c is attached to the third attachment site 110c in FIG. 5.

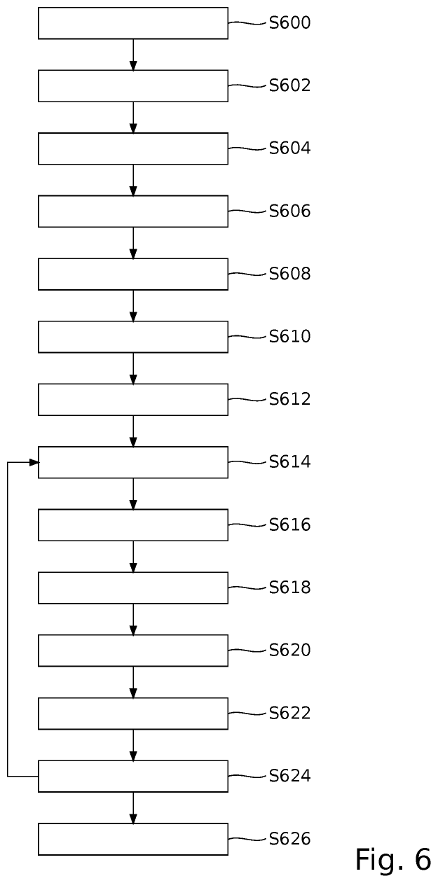

FIGS. 1 to 5 illustrate a method using the marker 100 for analyzing the biological sample 702 in which the three different reporters 114a, 114b, 114c are used to mark the predetermined structure 102 in a cyclical or round-based fashion. The method is described below with reference to FIG. 6. The method may be performed with a device that is described below with reference to FIG. 7.

FIG. 6 is a flowchart of the method for analyzing the biological sample 702 utilizing the marker 100 described above with reference to FIGS. 1 to 5.

The process is started in step S600. In step S602 at least one marker 100 as described above is provided. The markers 100 may for example be provided in a solution or a lyophilized solid. In particular, the marker base 104 and the different reporters 114a, 114b, 114c are provided separately.

In step S604 the marker bases 104 are introduced into the sample 702. The first reporters 114a may already be attached to the attachment structures 108 of the marker bases 104 when the marker bases 104 are introduced into the sample 702. Alternatively, the marker bases 104 and the first reporters 114a are introduced separately into sample 702. After the marker bases 104 had time to attach themselves to their respective predetermined structures 102 and the first reporters 114a had time to attach themselves to their respective marker bases 104, unbound marker bases 104 and reporters 114a, 114b, 114c are washed out of the sample 702 in an optional step S606. It may be the case that a specific predetermined structure 102 a marker 100 was supposed to attach itself to is not present in the sample 702. Washing out unbound marker bases 104 and reporters 114a, 114b, 114c thus ensures that only markers 100 that are actually attached to their associated predetermined structure 102 are detected in the following. It may also be the case, that not ever marker base 104 binds to its associated predetermined structure 102 or that not ever reporter 114a binds to its associated attachment site 110a, 110b, 110c. In this case, washing out unbound marker bases 104 and reporters 114a, 114b, 114c prevents misidentification of structures in the sample 702.

In step S608, at least one first excitation light is directed onto the sample 702 in order to excite the fluorescent dyes 120a, 120b, 120c, 120d, 120e of the first reporter 114a. The first excitation light may comprise light of a single wavelength or wavelength spectrum depending on the specific fluorescent dyes 120a, 120b, 120c, 120d, 120e of the first reporter 114a. More than one first excitation light may be used, for example light emitted by different light sources either simultaneously or sequentially. The excited fluorescent dyes 120a, 120b, 120c, 120d, 120e emit fluorescence light which is used to generate the at least one first readout in step S610. The first readout comprises information about the fluorescence light, in particular an emission spectrum, a fluorescence emission intensity or a fluorescence lifetime of the fluorescent dyes 120a, 120b, 120c, 120d, 120e. The information from the first readout is used to identify the fluorescent dyes 120a, 120b, 120c, 120d, 120e of the first reporters 114a, 114b, 114c, and thus the markers 100 present in the readout volume of the sample 702 in step S612. The steps S604 to S612 correspond to a first cycle or round of staining the sample 702 and imaging the sample 702.

Optionally, if every marker 100 in the sample 702 was identified with at least a predetermined certainty from the first readout alone, the process may be ended after step S612. Alternatively, the process is continued in step S614.

In step S614 the first cleaving agent 200 is introduced into the sample 702. The cleaving agents 200, 400 may be enzymatic cleaving agents in case the cleavage sites 112a, 112b are targets for said enzymatic cleaving agent. Alternatively, the cleaving agents 200, 400 may be light, in particular UV-light, in case the cleavage sites 112a, 112b can be cut by photolysis. After the attachment structure 108 has been cut between the first and second attachment sites 110a, 110b the removed first reporters 114a are washed out in step S616. In step 618 the second reporter 114b is introduced into the sample 702. In step 620 second excitation light is directed onto the sample 702 in order to excite the fluorescent dyes 120a′, 120b′, 120c′, 120d′, 120e′ of the second reporter 114b. The second excitation light may also comprise light of a single wavelength or wavelength spectrum depending on the specific fluorescent dyes 120a′, 120b′, 120c′, 120d′, 120e′ of the second reporter 114b. Likewise, more than one second excitation light may be used. In step 622 at least one second readout is generated from the fluorescence light emitted by excited fluorescent dyes 120a′, 120b′, 120c′, 120d′, 120e′ located in the readout volume of the sample 702. The information from the second readout is used to identify the fluorescent dyes 120a, 120b, 120c, 120d, 120e, 120a′, 120b′, 120c′, 120d′, 120e′ of second first reporters 114a, 114b and thus the markers 100 present in the sample 702 in step S624. The steps S614 to S624 correspond to a second cycle or round of staining the sample 702 and imaging the sample 702.

If every marker 100 in the sample 702 was identified with at least a predetermined certainty from the first and second readouts, the process is ended in step 626. Alternatively, the steps S614 to S624 are repeated for a third reporter 114c in a third cycle or round of staining the sample 702 and imaging the sample 702.

FIG. 7 shows a schematic drawing of a device 700 for analyzing a biological sample 702.

In particular, the device 700 is capable of performing the method for analyzing a biological sample 702 described above with reference to FIG. 6 utilizing markers 100 described above with reference to FIGS. 1 to 5. In Figure the device 700 is exemplary shown as being a part of a microscope system 704.

The device 700 comprises a staining unit 706 for introducing the marker 100 into the sample 702. For that purpose, the staining unit 706 may comprise one or more pipettes that may or may not be automated. The device 700 also comprises an excitation unit 708 for exciting the fluorescent dyes 120a, 120b, 120c, 120d, 120e of the different reporters 114a, 114b, 114c. The excitation unit 708 comprises at least one light source, preferably a coherent light source. The at least one light source is configured to emit the excitation lights used for exciting the fluorescent dyes 120a, 120b, 120c, 120d, 120e of the reporters 114a, 114b, 114c. In order to emit excitation light of different wavelengths or wavelength spectra, the light source may be a tunable light source. Alternatively, the device 700 may comprise two or more light sources with emitting light of different wavelengths or wavelength spectra. In the embodiment shown in FIG. 7, the excitation lights emitted by the excitation unit 708 is directed onto the sample 702 by a beam splitting unit 710.

An imaging unit 712 of the device 700 is configured to generate images from the fluorescence light emitted by the excited dyes. The images being the readouts in this embodiment. The imaging unit comprises an objective 714 directed at the sample 702 for capturing the fluorescence light. The captured fluorescence light is then directed onto a detection unit 716 by the beam splitting unit 710. The detection unit 716 comprises at least one detector element and a diffractive element for splitting the fluorescence light into different detection channels.

After generating a readout, the reporters 114a, 114b, 114c need to be removed from their respective markers 100 and the sample 702. This is done by means of the cleaving agent 200, 400. The cleaving agent 200, 400 may be enzymatic cleaving agent, which can be introduced into the sample 702 by means of the staining unit 706. Alternatively, the light source of the excitation unit 708 or an additional light source may be used to cut the attachment structures 108 by means of photolysis.

The device 700 further comprises a processor 718 connected to the staining unit 706, the excitation unit 708 and the detection unit 716. The processor 718 is configured to control the elements of the device 700 in order to perform the method for analyzing a biological sample 702. In particular, the processor 718 is configured to perform the method based on at least one user input.

Identical or similarly acting elements are designated with the same reference signs in all Figures. As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

LIST OF REFERENCE SIGNS

| | |
|---|---|
| 100 | Marker |
| 102 | Predetermined structure |
| 104 | Marker base |
| 106 | Affinity reagent |
| 108 | Attachment structure |
| 110a, 110b, 110c | Attachment site |
| 112a, 112b | Cleavage site |
| 114a, 114b, 114c | Reporter |
| 116 | Linker structure |
| 118a, 118b, 118c | Complementary attachment site |
| 120a, 120b, 120c, 120d, 120e, | Fluorescent dye |
| 120a′, 120b′, 120c′, 120d′, 120e′, | |
| 120a″, 120b″, 120c″, 120d″, 120e″ | |
| 200 | Cleaving agent |
| 400 | Cleaving agent |
| 700 | Device |
| 702 | Sample |
| 704 | Microscope system |
| 706 | Staining unit |
| 708 | Excitation unit |
| 710 | Beam splitting unit |
| 712 | Imaging unit |
| 714 | Objective |
| 716 | Detection unit |
| 718 | Processor |

1. A marker for marking a predetermined structure within a biological sample, the marker comprising:
a marker base having
an affinity reagent configured to attach to the predetermined structure of the sample, and
an attachment structure connected to the affinity reagent having at least two attachment sites,
wherein the attachment structure comprises at least one cleavage site arranged between the two attachment sites, and
wherein the attachment structure is capable of being cut at the at least one cleavage site by a cleaving agent in order to remove at least one attachment site from the marker base; and
at least two reporters, each reporter comprising
a linker structure having a complementary attachment site configured to attach to one of the two attachment sites of the attachment structure, and
a combination of at least two different fluorescent dyes arranged on the linker structure,
wherein the combination of the at least two different fluorescent dyes is unique for each reporter, and
wherein the complementary attachment site is unique for each reporter and configured such that each reporter attaches to a different attachment site of the marker base.

2. The marker according to claim 1, wherein the linker structures are formed by oligonucleotides or peptides.

3. The marker according to claim 1, wherein the cleavage site is an enzymatic cleavage site and the attachment structure is capable of being cut at the cleavage site by an enzymatic cleaving agent.

4. The marker according to claim 3, wherein the enzymatic cleavage site is a target site of a restriction enzyme, a CRISPR/Cas target, or a recombinase target site.

5. The marker according to claim 1, wherein the cleavage site is a photocleavage site, and the attachment structure is capable of being cut at the cleavage site by photolysis.

6. The marker according to claim 1, wherein the attachment sites and/or the complementary attachment sites are oligonucleotides.

7. A method for analyzing a biological sample, the method comprising:
providing at least one marker according to claim 1;
introducing the marker base and a first reporter of the at least two reporters into the sample;
directing at least a first excitation light onto the sample in order to excite the fluorescent dyes of the first reporter;
generating at least one first readout from fluorescence light emitted by the excited fluorescent dyes of the first reporter located in a readout volume of the sample;
introducing at least one cleaving agent into the sample in order to remove the first reporter from the marker base;
introducing a second reporter of the at least two reporters into the sample;
directing at least a second excitation light onto the sample in order to excite the fluorescent dyes of the second reporter;
generating at least one second readout from fluorescence light emitted by excited fluorescent dyes of the second reporter located in the readout volume of the sample; and
determining whether the marker is present in the readout volume based on the first readout and the second readout.

8. The method according to claim 7, further comprising cross-linking the affinity reagent of the marker to the predetermined structure with a cross-linking agent.

9. The method according to claim 7, wherein generating the first readout or the second readouts-readout comprises separating the emission light emitted by the excited fluorescent dyes into detection channels,
wherein the detection channels correspond to at least one emission characteristic of the fluorescent dyes, and
wherein the emission characteristic is one of: an emission spectrum, a fluorescence intensity, or a fluorescence lifetime.

10. The method according to claim 9, wherein the marker is configured such that each fluorescent dye of the first reporter and the second reporter corresponds to one detection channel of the first readout and the second readout, respectively.

11. The method according to claim 7, wherein the fluorescent dyes of the first reporter and the second reporter are divided into sets of fluorescent dyes;
wherein the fluorescent dyes in a same set are capable of being excited by a same wavelength or by a same wavelength spectrum;
wherein at least one of the first excitation light and the second excitation light is directed at the sample in order to excite the fluorescent dyes of the respective set;
wherein at least one of the first readout and the second readout is generated from the fluorescence light emitted by the respective set of fluorescent dyes located in the readout volume of the sample.

12. The method according to claim 11, wherein the first excitation light and the second excitation light are directed onto the sample in a sequence temporally following each other.

13. The method according to claim 7, wherein the first reporter is washed out of the sample before the second excitation light is directed onto the sample.

14. The method according to claim 7, wherein the first readout and/or the second readout comprises at least one image of the readout volume or a readout image data stream of the readout volume.

15. The method according to claim 7, further comprising capturing a hyperspectral image of the sample in order to generate the first readout or the second readout.

16. The method according to claim 7, further comprising stabilizing a fluorescence lifetime of at least one fluorescent dye, by placing the at least one fluorescent dye in a shielded environment by at least one of encapsulating, polymer-matrix embedding, or co-crystallizing.

17. A device for analyzing a biological sample being adapted to carry out the method according to claim 7.

18. The device according to claim 17, comprising a microscope, a plate reader, a cytometer, an imaging cytometer, or a fluorescence activated cell sorter configured to generate the first readout and the second readout.

19. The device according to claim 17, configured to determine at least one of: a fluorescence emission intensity, a fluorescence lifetime, a value representing a fluorescence lifetime, an emission spectrum, an excitation fingerprint, or a fluorescence anisotropy of the fluorescent dyes.
